(19) United States
(12) Patent Application Publication
(10) Pub. No.: US 2002/0010434 A1
(43) Pub. Date: Jan. 24, 2002

(54) Needle assembly with spring acted safety guard

(76) Inventors: Carsten Gerner Larsen, Lyngby (DK); Lars Morch Groth, Fredensborg (DK)

Correspondence Address: PATENT DEPARTMENT, SKADDEN, ARPS, SLATE, MEAGHER & FLOM LLP, FOUR TIMES SQUARE, NEW YORK, NY, 10036, US

(21) Appl. No.: 09/875,663

(22) Filed: Jun. 6, 2001

Related U.S. Application Data
(60) Provisional application No. 60/212,856, filed on Jun. 20, 2000.

(30) Foreign Application Priority Data
Jun. 9, 2000 (DK) PA 2000 00890

(51) Int. Cl. A61M 5/00; A61M 5/31

(52) U.S. Cl. 604/241; 604/198

(57) ABSTRACT

The needle assembly comprises a hub with a thread fitting the thread of a standard medical injection device. The hub is mounted with a cannula. The cannula has a proximal end that can penetrate the septum of a cartridge and a distal end that can penetrate the skin a patient. A protective sleeve surrounding the cannula is mounted at the distal end of a conical spring, which is mounted on the distal surface of the hub. The protective sleeve is locked in position near the distal surface of the hub. The protective sleeve is released when the needle is fully inserted into the patients skin, and slides along the needle cannula toward the distal end as the needle is withdrawn from the patient. When the protective sleeve reaches the distal end of the cannula it locks in position preventing further access to the distal end of the cannula.

The invention relates to a needle assembly, which reduced the risk of accidental needle-stick injuries, and especially safety needle assemblies where a needle cannula is mounted in a hub.

Needle assemblies are commonly used to both inject substances into and extract substances out of human and animal bodies. Such needle assemblies are typically disposable and are discarded after only one use. The problem presented by the disposal of a needle assembly, and indeed, by any handling of the needle assembly, is the potential risk for being injured by the sharp end of the needle. This is particular dangerous when following after the perforation of a patients skin since the needle assembly then may be contaminated and therefore capable of spreading diseases, such as hepatitis and HIV.

A great number of needle assemblies have been described that provide protection for the professionals who use injection needle assemblies in their daily work.

Such a prior art needle assembly is shown in WO 94/00172. This prior art needle assembly is made from a needle cannula supported by a hub being removable attached to an injection syringe. A guard is mounted on the hub and can be moved from a retracted position where the needle cannula can be used in a normal way to a second position where the guard covers the tip of the needle cannula thereby preventing accidental needle stick injuries. The guard surrounds the needle cannula and is provided with a channel only allowing passage of the needle cannula. A spring is provided between the guard and the hub, which spring forces the guard towards a position where the guard covers the tip of the needle cannula when the guard is released from its locked position. The locking mechanism is a single leaf-spring having a latching end, which is disengages when the needle cannula guard is forced against the skin of the patient, thereby releasing the guard.

These know needle assemblies are very cumbersome in their construction and have a large amount of movable parts. The fact that the known needle assembly is provided with only one leaf-spring holding the guard in the retracted position makes it very vulnerable to releasing the guard at the wrong time i.e. during the handling of the needle assembly, and once the guard has been released the needle assembly is no longer useable.

It is an object of the present invention to provide a needle assembly, which do not posses the drawbacks of the prior art needle assemblies, and where it is possible to handle the needle assembly rather roughly without releasing the guard from the retracted position.

This is obtained by a needle assembly with a needle cannula mounted in a hub for removable connection with an injection device, which needle cannula has a first distal end for piercing the skin of a patient and a second proximal end for entering said injection device, which needle assembly comprises;

a guard which can move relatively to the hub, the guard surrounds the needle cannula and has a channel or hole between a distal surface and a proximal surface of the guard allowing passage of the needle cannula, the guard is mounted upon the hub for linear movement relative thereto between a first position that permits normal use of the needle cannula and a second position in which at least the distal tip of the needle cannula is covered by the guard,

resilient means provided between the hub and the guard biasing the guard towards the second position when the guard is released from the hub, and

releasable locking means locking the guard onto said hub, 20

Which needle assembly according to the invention is characterized, in that the guard is provided with a number of flexible arms, which arms is locked in a number of apertures located on the hub when the guard is in the first position, and that the arms can be flexed by applying pressure on the guard thereby releasing the guard from the hub.

The flexible arms provided on the guard are locked in a number of apertures provided in the hub until an adequate pressure is applied onto the distal end of the guard. When the pressure presses the guard a short distance in the proximal direction the arms are moved into a position where the arms can flex in a way making it possible for the guard to move out of the locked position, when the applied pressure is released.

When, as disclosed in claim 2, the hub has a centrally located tower, which has a diameter substantially equal to the inside diameter of the guard, such that the guard can be fitted over the tower, it is ensured that the hub and the guard can be made very compact. The diameter of the tower and the width of the bended arms has to be fitted within the inside diameter of the guard in order to have both the guard and the bended arms slide over the rim of the hub-tower when the guard is released.

When, as disclosed in claim 3, the number of apertures located in the hub is an upper circumferential track and a lower circumferential track, which tracks preferably is located adjacent each other and connected to each other, and when, as disclosed in claim 4, the upper circumferential track is formed in the tower and has a bottom surface, which has a diameter smaller than the diameter of the tower, and, as disclosed in claim 5, the lower circumferential track is formed in the tower and has a bottom surface, which has a diameter being smaller than the diameter of the upper circumferential track, it is ensured that the apertures can both lock and release the arms of the guard in a suitable manner.

When, as disclosed in claim 6 each of the flexible arms provided on the inside surface of the guard has a length X in the radial direction, where X is larger than half the difference between the diameter of the tower and the diameter of the bottom surface of the upper track, and X is smaller than half the difference between the diameter of the tower and the diameter of the bottom surface of the lower track, it is ensured that the arms are too long to fit into the upper track in aligned condition, but can be fitted in the lower track. The arms are therefore bended and locked when positioned in the upper track, but are aligned when positioned in the lower track thereby making it possible to bend the arms backwards.

When, as disclosed in claim 7, the resilient means is a spring surrounding the tower and being connected to the hub and to the guard, it is ensured that a sufficient force can be applied on the guard when the guard is released from the first position.

When, as disclosed in claim 8, the arms is bended and locked in the first circumferential track when the guard is in the first position, it is ensured that the guard is secured in the first position when the needle assembly is not in use.

When, as disclosed in claim 9, the arms are moved from the upper track to the lower track when pressure is applied onto the distal end of the guard, and when, as disclosed in claim 10, the arms have an inherent resilience, which aligns the arms when they are positioned in the second circumferential track, thereby allowing the guard to move forward relatively to the hub, it is ensured that the guard is released and moved to the second position when the needle assembly is being used.

The invention will be explained more fully below in connection with a preferred embodiment and with reference to the drawings in which:

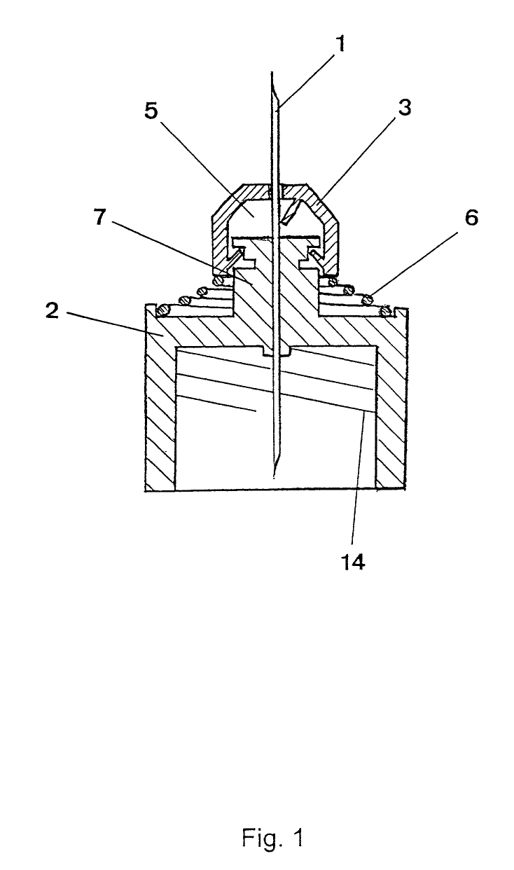

FIG. 1 Shows a sectional view of the needle assembly according to the invention prior to use.

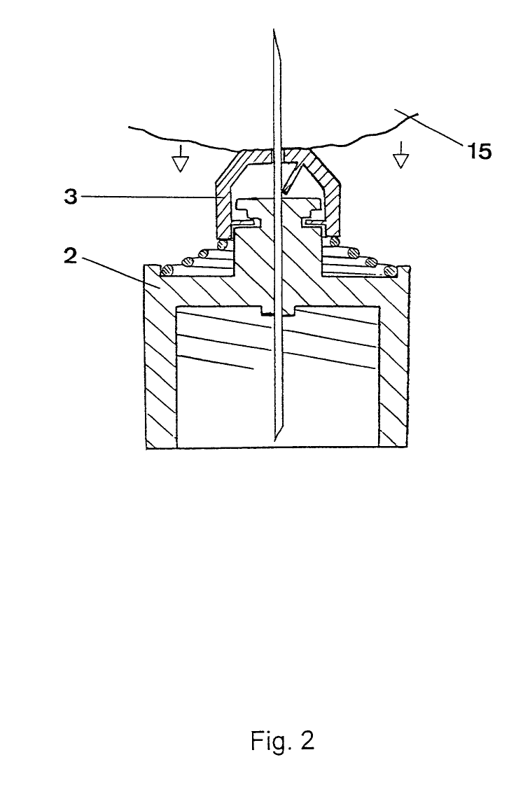

FIG. 2 Shows a sectional view of the needle assembly according to the invention in use

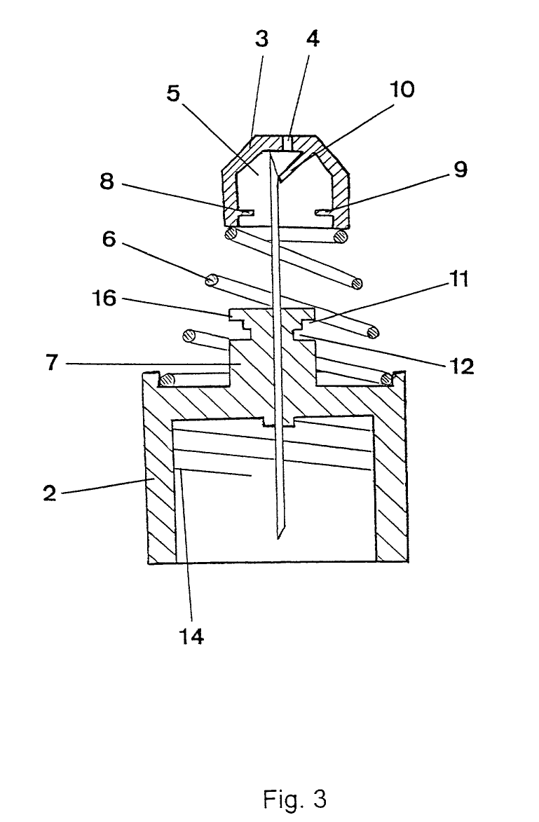

FIG. 3 Shows a sectional view of the needle assembly according to the invention after use.

The figures are schematic and simplified for clarity, and they just show details, which are essential to the understanding of the invention, while other details are left out. Throughout, the same reference numerals are used for identical or corresponding parts.

FIG. 1 shows the needle assembly prior to use. The needle cannula 1 is mounted in the hub 2. The needle cannula 1 has a distal end for piercing the skin of a patient and a proximal end for piercing the elastomeric seal of a cartridge carried in the injection device onto which the needle assembly is mounted.

The guard 3 has at the distal end a tiny hole or channel 4, which allows passage of the needle cannula 1. At the proximal end the guard 3 is provided with a larger hole or cavity 5 into which a tower 7 centrally located on the hub 2 is fitted.

The inside surface of the guard 3 is provided with a number of flexible arms 8, 9, which arms 8, 9 locks the guard 3 onto the hub 2 as will be explained later. The inside surface of the guard 3 is also provided with a flexible flap 10, which flap 10 is cocked when the needle cannula 1 is guided in the hole or channel 4 of the guard 3 and which flap 10 slides along the needle cannula 1 and urges the guard 3 sideways when the needle cannula 1 is no longer guided in the hole or channel 4 as shown in FIG. 3, thereby preventing reuse of the needle assembly.

The tower 7 of the hub 2 is provided with two circumferential depressions or tracks 11, 12. The bottom surface of the upper track 11 has a diameter smaller than the diameter of the tower 7, leaving a rim 16 at the distal end of the tower 7. The lower track 12 is located adjacent the upper track 11 and the two tracks 11, 12 is connected to each other. The lower track 12 has a bottom surface with a diameter yet smaller than the diameter of the bottom surface of the upper track.

A spring 6, e.g. a helical or a conical spring, surrounding the needle cannula 1 and the tower 7 is positioned between the hub 2 and the guard 3 urging the guard 3 away from the hub 2. The spring 6 is connected both to the hub 2 and to the guard 3, and has an inherent resilience and a length, which positions the guard 3 around the tip of the needle cannula 1 when the guard is released from the hub 2.

In use the needle assembly is first mounted onto the not shown injection device e.g. by screwing the hub onto the injection device utilising the thread 14 of the hub 3.

The flexible arms 8, 9 located on the inside surface of the guard 3 has a radial length making it impossible to house the arms 8, 9 in their aligned position inside the upper track 11. Both arms 8, 9, are therefore bended a little into an upright position i.e. pointing towards the distal end of the needle assembly, which locks the arms in the upper track 11 of the hub-tower 7. In this position the needle cannula 1 is exposed and ready for injection, as shown in FIG. 1

It is to be understood that the arms 8, 9 can either be provided as a number of individual arms 8, 9 located along the inside surface of the guard 3, or the arms 8, 9 can be provided as one circular skirt provided on the inside surface of the guard 3.

During injection the guard 3 is forced against the skin 15 of the patient as shown in FIG. 2. This forces the guard 3 to move relatively to the hub 2 in the proximal direction, during this movement the flexible arms 8, 9 will, when located in the lower track 12, assume there aligned position, which is also shown in FIG. 2. When the needle cannula 1 is retracted from the patients skin 15 and the force on the guard 3 is released, the arms 8, 9 now being in their aligned position will bend in the proximal direction and allow arms 8, 9 to bend over the rim 16 of the upper track 11 thereby allowing the guard 3 to move to a position where the guard 3 covers the tip of the needle cannula 1 as shown in FIG. 3.

The diameter of the tower 7, the diameter of the rim 16, the inside diameter of the guard 3 and the width of the arms 8, 9 has to be dimensioned such that the guard and the bended arms 8, 9 provided on the inside surface of the guard 3 can pass over the rim 16 when the guard 3 is moving from the first position to the second position.

A preferred embodiment has been shown in the foregoing, but it should be stressed that the invention is not limited to these, but may be embodied in other ways within the subject matter defined in the following claims. The flap 10 could for example be replaced by some other mechanism preventing reuse of the needle assembly. The mechanism could be a lanyard connecting the hub 2 and the guard 3 in a displaced position, which would cause the guard 3 to be pulled away from the centre-line when the lanyard is stretched and the needle cannula is free of the hole 4 in the guard 3.

1. A needle assembly with a needle cannula mounted in a hub for removable connection with an injection device, which needle cannula has a first distal end for piercing the skin of a patient and a second proximal end for entering said injection device, said needle cannula comprising
a guard which can move relatively to said hub, said guard surrounding said needle cannula and having a channel or hole between a distal surface and a proximal surface of said guard allowing passage of said needle cannula, said guard being mounted upon said hub for linear movement relative thereto between a first position that permits normal use of said needle cannula and a second position in which at least the distal tip of said needle cannula is covered by said guard,
resilient means provided between said hub and said guard biasing said guard towards said second position when said guard is released from said hub, and
releasable locking means locking said guard on to said hub,
characterized, in that said guard is provided with a number of flexible arms, which arms is locked in a number of apertures located on said hub when said guard is in said first position, and that said arms can be flexed by applying pressure on said guard thereby releasing said guard from said hub.

2. A needle assembly according to claim 2, characterized in that said hub has a centrally located tower, which has a diameter substantially equal to the inside diameter of said guard, such that said guard can be fitted over said tower.

3. A needle assembly according to claim 1 or 2, characterized in that said number of apertures located in said hub is an upper circumferential track and a lower circumferential track, which tracks preferably is located adjacent each other and connected to each other.

4. A needle assembly according to claim 3, characterized in that said upper circumferential track is formed in said tower and has a bottom surface, which has a diameter smaller than the diameter of said tower.

5. A needle assembly according to claim 3, characterized in that said lower circumferential track is formed in said tower and has a bottom surface which has a diameter being smaller than the diameter of said upper circumferential track.

6. A needle assembly according to anyone of claims 3 to 5, characterized in that each of said flexible arms provided on the inside surface of said guard has a length X in the radial direction, where;
X is larger than half the difference between the diameter of said tower and the diameter of said bottom surface of said first track, and
X is smaller than half the difference between the diameter of said tower and the diameter of said bottom surface of said second track.

7. A needle assembly according to anyone of the preceding claims, characterized in that said resilient means is a spring surrounding said tower and being connected to said hub and to said guard.

8. A needle assembly according to claim 6 or 7, characterized in that said arms is bended and locked in said first circumferential track when said guard is in said first position.

9. A needle assembly according anyone of the claims 3-8, characterized in that said arms is moved from said upper track to said lower track when pressure is applied onto the distal end of said guard.

10. A needle assembly according to claim 3-9, characterized in that said arm has an inherent resilience which aligns said arms when they are positioned in said second circumferential track, thereby allowing said guard to move forward relatively to said hub.
